(19) United States
(12) Patent Application Publication
(10) Pub. No.: US 2017/0360581 A1
(43) Pub. Date: Dec. 21, 2017

(54) RESPONSIVE PROSTHESES

(71) Applicant: Baylor College of Medicine, Houston, TX (US)

(72) Inventors: Jared Howell, Manvel, TX (US); Lorin Merkley, Pearland, TX (US)

(73) Assignee: Baylor College of Medicine, Houston, TX (US)

(21) Appl. No.: 15/525,482

(22) PCT Filed: Nov. 30, 2015
(86) PCT No.: PCT/US2015/063041

§ 371 Date: May 9, 2017

Related U.S. Application Data
(60) Provisional application No. 62/087,031, filed on Dec. 3, 2014.

(51) Int. Cl. A61F 2/66; A61F 2/50

(52) U.S. Cl. CPC A61F 2/66; A61F 2002/6678; A61F 2002/6671; A61F 2002/6642; A61F 2002/5006; A61F 2002/5033; A61F 2002/5007; A61F 2002/6621

(57) ABSTRACT

One aspect of the invention provides a prosthetic foot including a heel member, a toe member, and an attachment member comprising a shear thickening material (STM). In one embodiment, the attachment member has a first end and a second end. The first end can be connected to the heel member, and the second end can be connected to the toe member. In another embodiment, the attachment member further comprises an elastic polymer. In one instance, the elastic polymer is impregnated with the STM. In yet another embodiment, the attachment member further comprises an elastic reservoir. The elastic reservoir can contain the STM. Alternatively, the attachment member further comprises a rigid reservoir. The rigid reservoir can contain the STM.

### CROSS-REFERENCE TO RELATED APPLICATION

This application claims priority to U.S. Provisional Patent Application Ser. No. 62/087,031, filed Dec. 3, 2014. The entire content of this application is hereby incorporated by reference herein.

### BACKGROUND OF THE INVENTION

Prostheses are artificial limbs and/or body parts that aim to help an amputee regain normal body function.

### SUMMARY OF THE INVENTION

One aspect of the invention provides a prosthetic foot including a heel member, a toe member, and an attachment member comprising a shear thickening material (STM).

In one embodiment, the attachment member has a first end and a second end. The first end can be connected to the heel member, and the second end can be connected to the toe member.

In another embodiment, the attachment member further comprises an elastic polymer. In one instance, the elastic polymer is impregnated with the STM.

In yet another embodiment, the attachment member further comprises an elastic reservoir. The elastic reservoir can contain the STM. Alternatively, the attachment member further comprises a rigid reservoir. The rigid reservoir can contain the STM.

In yet another embodiment, the attachment member is the lamina of the prosthetic foot. In yet another embodiment, the attachment member further comprises a rigid sealed container containing the STM and a piston. The piston is connected to the heel member.

The STM used in the prosthetic foot of the present invention improves the shock absorption, weight bearing, and modulation of the prostheses. In one embodiment, the STM is a mixture of silica and polyethylene glycol. In another embodiment, the STM is selected from silicone polymers exhibiting shear thickening properties. The silicone polymer can be selected from borated silicone polymers. In one instance, the borated silicone polymer is polyborodimethylsiloxane (PBDMS).

The attachment member of a prosthetic foot of the invention can comprise 50-100% STM by weight. In one embodiment, the attachment member comprises 65-95% STM by weight. In another embodiment, the attachment member comprises 70% STM by weight.

The prosthetic foot of the present invention further comprises an outer shell.

### BRIEF DESCRIPTION OF THE DRAWINGS

For the purpose of illustrating the invention, certain embodiments of the invention are depicted in the drawings. However, the invention is not limited to the precise arrangements and instrumentalities of the embodiments depicted in the drawings.

FIG. 1 is a set of images depicting the stages of gait while walking.

FIG. 2 depicts a base built to hold the prostheses during trials. Four 2.5″×3.5″×14″ wood supports were attached to each side of a 3.54″×3.54″ wood post at the base using wood glue and screws. The wood supports were cut equal in length and all rough edges sanded. A 3.5″×3.5″ aluminum metal plate was screwed down to the top of the 3.54″×3.54″ wood post with a 4″ lag bolt in the center.

FIG. 3 depicts the complete setup for the trials. A prosthetic pyramid was connected to the metal plate on the base, and a prosthetic pylon was attached to the pyramid. A white board made from plywood that was painted white with black lines spaced 0.25″ apart was attached to the back of the 3.54″×3.54″ wood post. A yard stick was attached to the front of the wood post. A 0.5″ PETG spacer was placed between the yard stick and the wood post to prevent the yard stick from interfering with the squat bar. The bottom screw was secure and the top screw was covered with a washer and removable nut so that the yard stick could be pulled off the top screw and laid to the side while changing out the prosthetic foot. A high speed camera was used to record data.

FIG. 4 is a graph depicting the deflection of a NITRO® prosthetic running foot when the barbell falls at different heights.

FIG. 5 is a graph depicting the energy return relative to deflection of NITRO® prosthetic running foot. Diamonds represent the NITRO® prosthetic running foot without an attachment member comprising a STM. Circles represent the NITRO® prosthetic running foot with an attachment member comprising a light stiffness shear thickening material (STM) attached at the angle of maximum compression.

FIG. 6 is a graph depicting the deflection of a RUSH® 87 prosthetic foot when the barbell falls at different height.

FIG. 7 is a graph depicting the energy return relative to deflection of a RUSH® 87 prosthetic foot. Diamonds represent the RUSH® 87 prosthetic foot with an attachment member comprising a medium stiffness STM. Circles represent the RUSH® 87 prosthetic foot with an attachment member comprising a high stiffness STM.

FIG. 8 is a graph depicting the deflection of a RENEGADE® prosthetic foot when the barbell falls at different height.

FIG. 9 is a graph depicting the energy return relative to deflection of the RENEGADE® prosthetic foot. Diamonds represent the RENEGADE® prosthetic foot without an attachment member comprising a STM. Circles represent the RENEGADE® prosthetic foot with an attachment member comprising a medium stiffness STM.

FIG. 10 is an image depicting an attachment member comprising a STM attached to a prosthetic foot using a duct tape.

FIG. 11 is an image depicting an attachment member comprising a STM attached only to the heel member of a prosthetic foot.

FIG. 12 is an image depicting an attachment member comprising a STM, a piston, and a rigid sealed container/reservoir. The attachment member is connected to the top of heel member in one end and to the bottom of heel member in another end.

FIG. 13 is an image depicting an attachment member comprising a flexible reservoir containing a STM. The attachment member is positioned between the heel member and the toe member of a prosthetic foot.

FIG. 14 is an image depicting an attachment member comprising a STM incorporated into a prosthetic foot as an integral part of the prosthetic foot.

FIG. 15 is an image depicting the incorporation of STM within a torsion adaptor to provide additional damping.

FIG. 16 is an image depicting the incorporation of STM within a torsion adaptor to slow rotation due to the shear thickening effect.

FIG. 17 is an image depicting the potential use of STM in different parts of prosthetic legs and feet.

FIG. 18 is an image depicting a molded foot with a STM outer shell.

FIG. 19 is a graph depicting the compressibility of different STMs at varying impact speeds. A pendulum with an impact arm at the end was raised to a predetermined height and then released. The pendulum would swing and impact the STMs. The starting pendulum height was adjusted to recreate impact speeds of 0.8 m/s, 1.57 m/s, 1.96 m/s, 2.35 m/s, and 2.89 m/s. The pendulum was dropped without any excess weight or with a 30 lb lead block attached on the end of the pendulum.

### DEFINITIONS

As used herein, each of the following terms has the meaning associated with it in this section.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

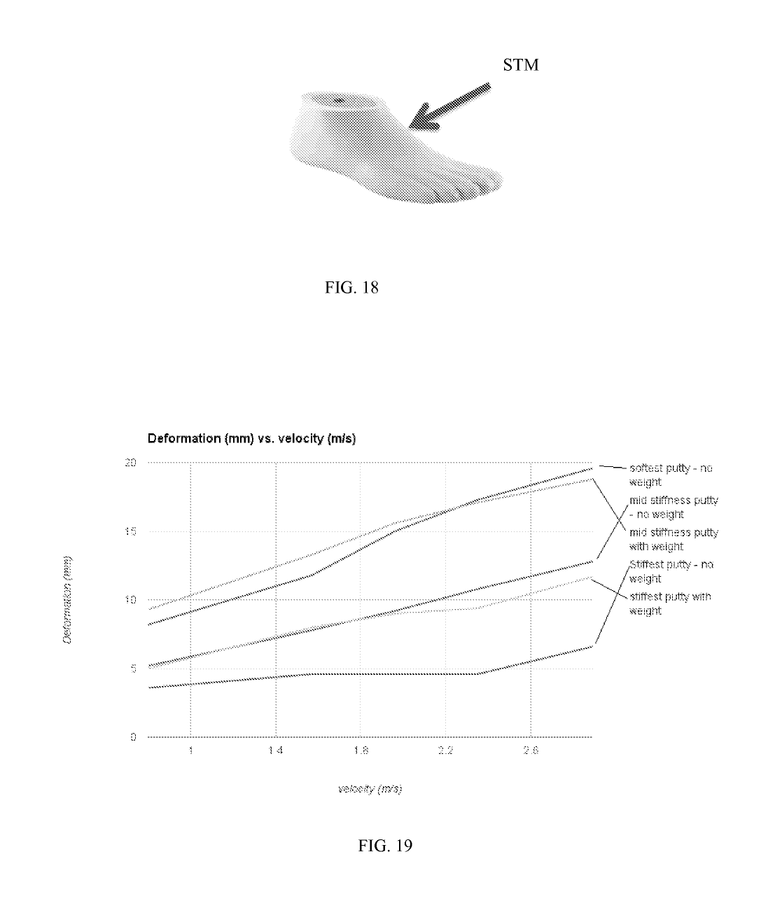

As used herein, the term “attachment member” refers to a structure comprising of a STM, which can be a component of a prosthetic device. For example, the attachment member can be attached to or connected to either heel member or toe member or both. Alternatively, the attachment member can be attached to or connected to either the vicinity of heel member or vicinity of toe member or both. In another example, the attachment member can be the outer shell of a molded foot (FIG. 18).

As used herein, “dilatant,” “shear thickening material,” and “shear thickening fluid” are used interchangeably. All refer to a material in which viscosity increases based on shear strain. It is a material that stiffens proportional to the impact placed on it.

As used herein, the term “heel member” refers to a part of the structure of a prosthetic foot simulating the function of natural a heel. The heel member can be rigid or flexible and may or may not incorporate a mechanical lever.

As used herein, “NITRO®” refers to a prosthetic foot made by Freedom Innovations, Irvine, Calif.

As used herein, “RENEGADE®” refers to a prosthetic foot made by Freedom Innovations, Irvine, Calif.

As used herein, “RUSH® 87” refers to a prosthetic foot made by Ability Dynamics, Tempe, Ariz.

As used herein, the term “toe member” refers to a part of the structure of a prosthetic foot simulating the function of natural toes. The toe member may also be termed the “keel” of the prosthetic foot.

### DETAILED DESCRIPTION OF THE INVENTION

One aspect of the present invention incorporates a shear thickening material (STM) in limb prostheses, which includes upper extremity prostheses and lower extremity prostheses. Upper extremity prostheses are used at varying levels of amputation and include forequarter, shoulder disarticulation, transhumeral prosthesis, elbow disarticulation, transradial prosthesis, wrist disarticulation, full hand, partial hand, finger, partial finger.

Lower extremity prostheses provide replacements at varying levels of amputation and include hip disarticulation, transfemoral prosthesis, knee disarticulation, transtibial prosthesis, Syme's amputation, foot, partial foot, and toe.

### Shear-Thickening Materials (STMs)

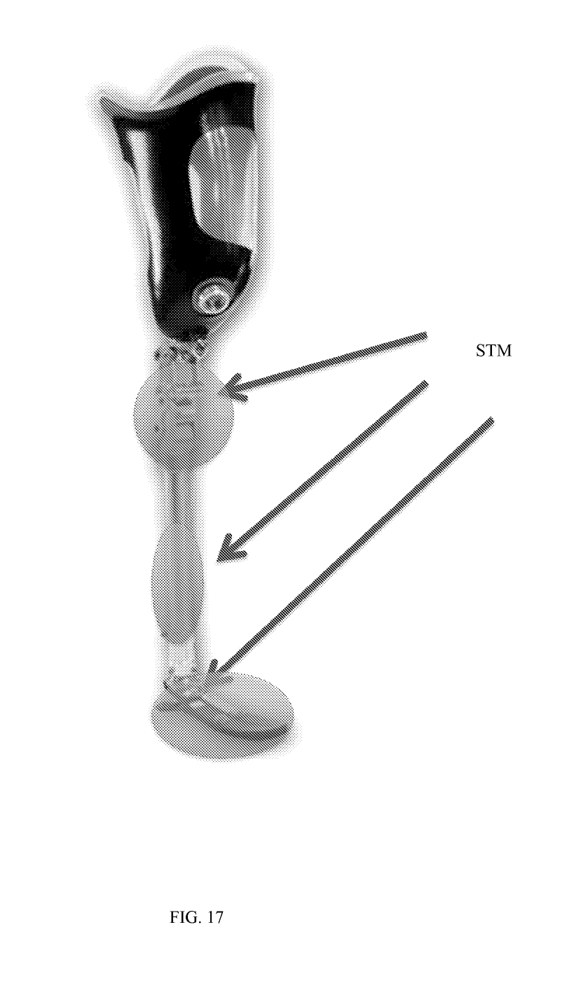

A STM can be incorporated into prostheses at various positions as needed (FIG. 17). Incorporation of a STM can improve the shock absorption, weight bearing, and modulation of the prostheses. STM is a material in which viscosity increases based on shear strain. It is a material that stiffens proportional to the impact placed on it. Under a hard forceful blow, the STM stiffens and resists deformation. Under lower forces, the STM is more compliant. The STM can be in the form of a liquid, gels, polymers or putties, depending on the need of the prostheses.

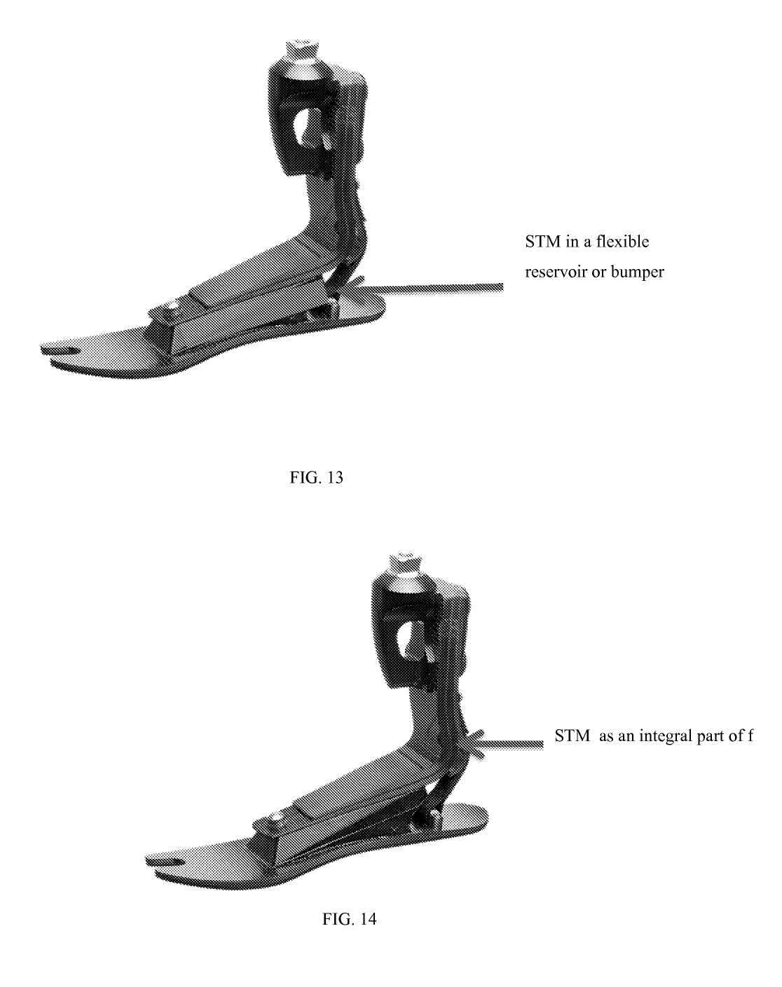

In one embodiment, the STM would be contained within a confined but elastic reservoir. This reservoir can be strategically placed within the structure of a prosthetic foot so that by it being compressed and elongated, similar to a bumper, the rigidity and elasticity of the overall foot would be changed according to the instantaneous stiffness of the reservoir (FIG. 13).

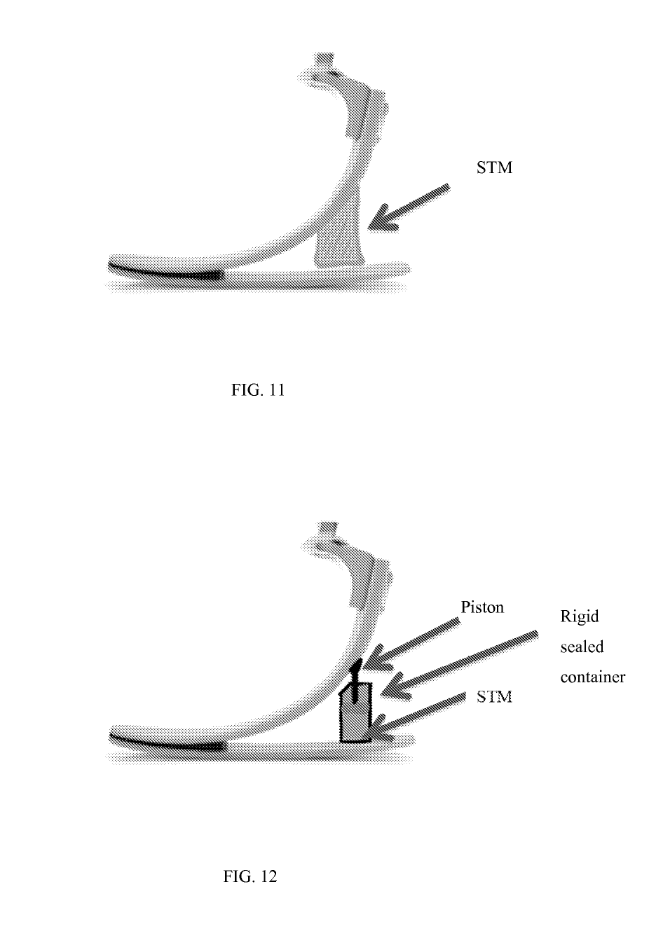

In another embodiment, the STM can be used by itself in polymer form or in combination with other substrate create a molded bumper (FIG. 11).

In another embodiment, the STM can be fabricated to make up the lamina of the prosthetic foot and is an integral part of the prosthetic foot (FIG. 14).

In another embodiment, the STM can be placed within a rigid sealed container/reservoir with members that can flow through the fluid within. These members may be similar but not limited to a piston, chords, or webbing. As the member passes through the fluid the resistance on the member would be proportional to the speed of the passing motion (FIG. 12).

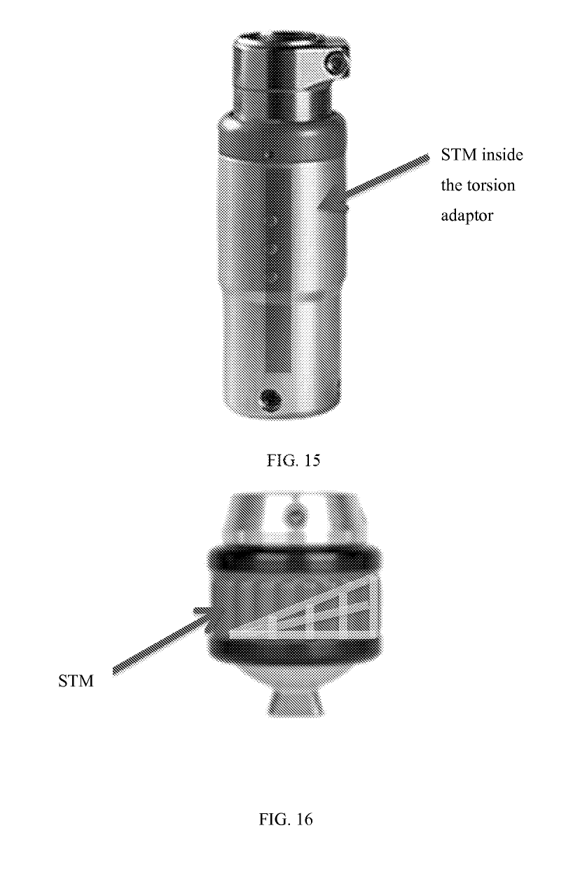

In further embodiments, an STM in form of a liquid inside a torsion adaptor can be used to modulate the speed of rotation and provides additional damping (FIG. 15). A STM in the form of polymer, putty or gel inside a torsion adaptor can increase the stability and slow rotation (FIG. 16). Also, a STM can be used in prostheses to provide proportional resistance to forces at knee, foot, and torsion components (FIG. 17).

The STM described herein can be a polymer or a composition exhibiting shear thickening properties. Various STMs are described in Zhao, et al., 2013, Adv. Colloid Interface Sci. 201-202:94-108). Polymerized STMs are available from D30 Lab of East Sussex, United Kingdom and ZB Products, L.P. of Katy, Tex.

In one embodiment, the STM is selected from silicone polymers exhibiting shear thickening properties. In another embodiment, the STM is selected from borated silicone polymers. In another embodiment, the STM is polyborodimethylsiloxane. Examples of STMs and their properties are disclosed in U.S. Pat. No. 7,381,460.

In another embodiment, the STM is so-called “silly putty”, made from silicone oil and boric acid. In one instance, the STM is a mixture of silica nano-particles dispersed in a solution of polyethylene glycol. In another instance, the STM comprise 20-80% silica nanoparticles by weight dispersed in 200 MW polyethylene glycol.

In some embodiments, the STM is a non-magnetorheological fluid. Such a fluid would exhibit variable damping properties without the need to apply a magnetic field to the fluid as is the case with magnetorheological fluids.

### Prosthetic Feet

Another aspect of the present invention relates to a new and responsive prosthetic foot comprising a STM. The prosthetic foot described herein adapts to changes in loading (e.g., running vs. walking). In return, it would allow feet to adapt the user's demands rather than requiring the user to adapt the foot for every change in load or impact. In other words, the prosthetic foot would allow the individuals with amputation to have a prosthesis that automatically adapts itself for optimal stiffness based on the demands placed on it. The prosthetic foot described herein has the ability to function under different physical demands due to adaptation to outside stresses.

In one embodiment, the prosthetic foot comprises a heel member, a toe member, and an attachment member.

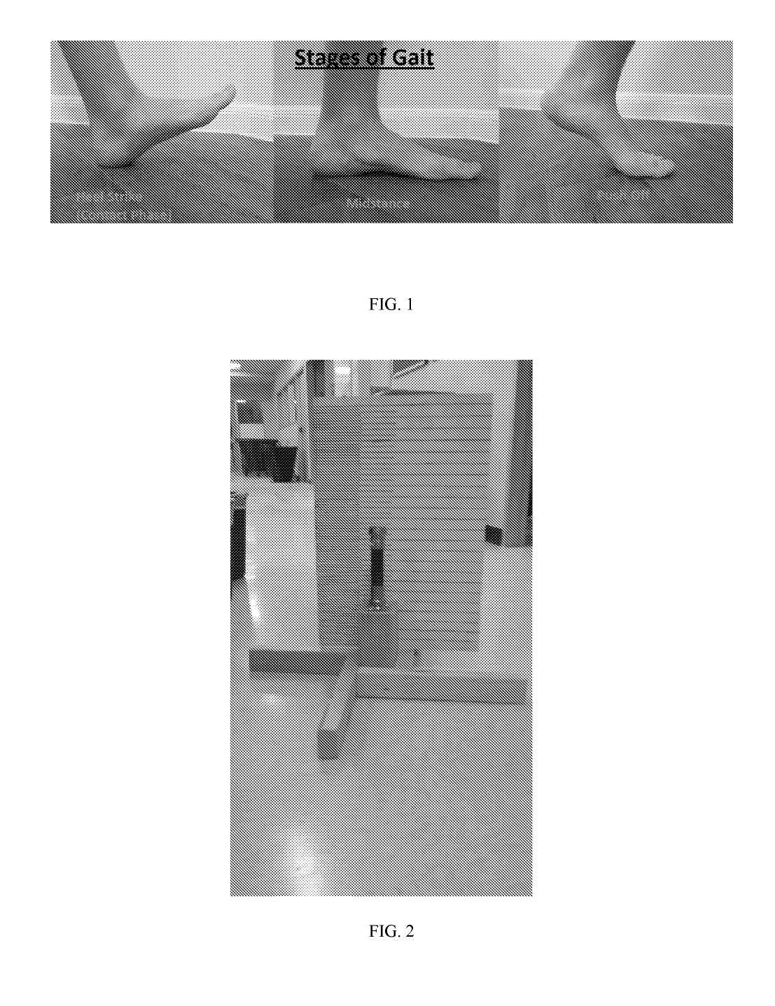

A heel member of a prosthetic foot simulates a natural ankle and functions to absorb shock, bear weight, and ambulate. A toe member of a prosthetic foot simulates the toes of a natural foot to push off while walking (FIG. 1). The attachment member comprises a STM and maximizes shock absorption and energy return when walking. The attachment member is the key element enabling the prosthetic foot to automatically adapt itself for optimal stiffness based on the demands placed on it.

In one embodiment, the attachment member has a first end and a second end. In one instance, the first end is connected the heel member and the second end is connected to the toe member (FIG. 10). In another instance, the first end is connected the heel member while the second end is not connected to any part of the prosthetic foot (FIG. 11). As used herein, the term “connected” refers to formation of a connection using a fastener, a tape, an adhesive, or a joint to physically join two parts together. In another instance, the attachment member is placed between the heel member and toe member (FIG. 13).

In certain embodiments, the attachment member further comprises an elastic polymer, and the elastic polymer is impregnated with a STM. The elastic polymer can be natural elastomers, e.g. latex rubbers or synthetic elastomers, including synthetic thermoplastic elastomers, e.g. elastomeric polyurethanes. In one instance, the attachment member comprises a STM and an elastic polymer.

In another embodiment, the attachment member comprises an elastic polymer and a reservoir. The reservoir is enclosed by the elastic polymer and contains a STM. In yet another embodiment, the attachment member comprises a rigid sealed container/reservoir containing a STM and a piston like structure moving within the container (FIG. 12).

In any of these embodiments, one or more components of the attachment member can be configured to provide resilient deformability so that deformation of the STM during low loads will be completely or substantially completely reversed after the load is removed.

In the case of the polymerized STM, the STM itself provides this resilient deformability. In other embodiments, one or more additional members provides the desired resiliency. For example, an elastic reservoir (e.g., a rubber boot or surgical tubing) can squeeze against the STM contained therein to return the STM to its original shape. Springs in an STM-containing shock absorber or textiles impregnated with an STM can provide the same effect.

The elasticity of the additional members can be calibrated to provide a desired level of deformation under low level loads while maintain sufficient resilient strength to urge the STM back to its original position once the load is removed.

In yet another embodiment, the attachment member comprises about 30-100%, 50-100%, or 65-95% by weight of a STM. In one instance, the attachment member comprises 70% by weight of a STM.

In yet another embodiment, the attachment member can be added or incorporated into an existing prosthetic foot. Alternatively, the attachment member can be fabricated together with a new prosthetic foot.

In yet another embodiment, the prosthetic foot further comprises an outer shell. The outer shell can be fabricated in the shape of a foot so that a shoe can be worn over the prosthetic. In one instance, the attachment member is the outer shell of molded foot (FIG. 18).

### WORKING EXAMPLES

The invention is further described in detail by reference to the following working examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Although the following examples demonstrate the incorporation of an attachment member comprising a STM into an existing prosthetic foot, it is well understood that such attachment member can also be incorporated into a new prosthetic foot.

### Materials

### Study Design

This study used three different types of prosthetic feet: the NITRO® foot, the RUSH® 87 foot, and the RENEGADE® foot. No human subjects were involved; therefore, no Institutional Review Board (IRB) approval was required.

### Foot Prosthesis Selection

Three different foot prosthesis models were selected for this study: the NITRO® running foot, the RUSH® 87 foot, and the RENEGADE® foot. The NITRO® running foot demonstrates maximal vertical displacement (i.e. ability to compress), the RUSH® walking foot also possesses great flexibility, and anecdotal clinical evidence suggests significant vertical displacement in the heel of a Freedom Innovations RENEGADE® prosthetic foot. These three models of prosthetic feet chosen are ideal for this study because their flexibility and ability to compress will allow the most changes to be seen when an attachment member comprising a STM is attached.

### Building the Base

A base was built to hold the prostheses during trials. Four 2.5″×3.5″×14″ wood supports were attached to each side of a 3.54″×3.54″ wood post at the base using wood glue and screws. The wood supports were cut equal in length and all rough edges sanded.

A 3.5″×3.5″ aluminum metal plate was screwed down to the top of the 3.54″×3.54″ wood post with a 4″ lag bolt in the center (FIG. 2). A prosthetic pyramid was connected to the metal plate, and a prosthesis pylon was attached to the pyramid. A white board made from plywood that was painted white with black lines spaced 0.25″ apart was attached to the back of the 3.54″×3.54″ wood post. A yard stick was attached to the front of the wood post.

A 0.5″ PETG spacer was placed between the yard stick and the wood post to prevent the yard stick from getting in the way of the squat bar (FIG. 3). The bottom screw was secured and the top screw was covered with a washer and removable nut so that the yard stick could be pulled off the top screw and laid to the side while changing out the prosthetic foot.

### Methods

### 1. Data Collection

Each model of prosthetic foot was attached to the wooden base one at a time. The RUSH® 87 and RENEGADE® feet were positioned anteriorly so that the bar would land squarely on the heel of the prosthesis. The RUSH® 87 and RENEGADE® were kept attached to their corresponding tube clamp adapter when changing the prostheses to maintain consistency in their angle. A colored dot was placed on each foot prostheses where most deflection would occur so that deflection would be easily seen on camera and calculated. The wooden base was placed so that the prosthesis was underneath and parallel to the squat bar to allow visibility of displacement by the camera. The bar would be lowered onto the prosthesis before beginning trials to make sure it landed in the middle of the prosthesis. Once there was good placement of the prosthesis on the wooden base, the base was secured to the floor using duct tape to help further stabilize the structure. The GOPRO® Hero 3+ Black Edition high speed camera (240 fps) was placed anterior to the squat rack. The GOPRO® phone application was used to make sure good position of the camera had been obtained. 25 lb and 2.5 lb weights were added to each end of the squat bar for a total of 100 lbs. A nylon rope was used to help aid in lifting the squat bar. The nylon rope was tied to each end of the squat bar then thrown over the top of the machine to create a pulley system. The squat rack bar was then dropped from 4″, 8″, and 12″ for three trials at each height for each foot prosthesis with and without an attachment member comprising an STM while being recorded by the camera. The attachment member comprising a STM was added to each prosthesis and secured using duct tape. On the NITRO® running foot, the attachment member comprising a STM was placed between the two points of maximal compression. Only the attachment member comprising a “light” stiffness STM was trialed on the NITRO® running foot. On the RUSH® 87 foot, the attachment member comprising a STM was placed inside the hollowed out bumper. Both the attachment member comprising “middle” and “high” stiffness STMs were trialed on the RUSH® 87. On the RENEGADE® foot, the attachment member comprising a STM was placed between each of the ridges in the heel. The RENEGADE® was tested with both the attachment member comprising “light” and “middle” stiffness STMs.

### 2. Statistical Analysis

The videos were analyzed to calculate the amount of vertical displacement. This was calculated by measuring the change in height from the prosthesis at rest to maximum deflection during each trial. Energy return was analyzed by measuring the height the squat bar bounced off the prosthesis, starting the measurement from maximum deflection. IBM SPSS® statistical software, version 22, was used to calculate parametric data.

### 3. Results

Table 1 shows the comparison of mean deflection pre and post addition of an attachment member comprising a STM. This table shows that there is a statistical significance in the amount of decreased deflection in the NITRO® prosthetic and RENEGADE® prosthetic and in the increased deflection in the RUSH® 87 prosthetic.

TABLE 1
Comparison of mean deflection pre and post addition of STM

| | | | Significance |
|---|---|---|---|
| Prosthesis | Category | Mean (in inches) | (2-tailed parametric) |
| Nitro | Pre 4″ | 2.1 | 0.000 |
| | Post 4″ | 1.79 | |
| | Pre 8″ | 2.52 | 0.000 |
| | Post 8″ | 2.21 | |
| | Pre 12″ | 2.85 | 0.000 |
| | Post 12″ | 2.52 | |
| Rush | Pre 4″ | 0.75 | 0.000 |
| | Post 4″ | 1.19 | |
| | Pre 8″ | 0.875 | 0.000 |
| | Post 8″ | 1.31 | |
| | Pre 12″ | 0.92 | 0.000 |
| | Post 12″ | 1.375 | |
| Renegade | Pre 4″ | 0.69 | 0.000 |
| | Post 4″ | 0.5 | |
| | Pre 8″ | 0.75 | 0.000 |
| | Post 8″ | 0.625 | |
| | Pre 12″ | 0.79 | 0.000 |
| | Post 12″ | 0.69 | |

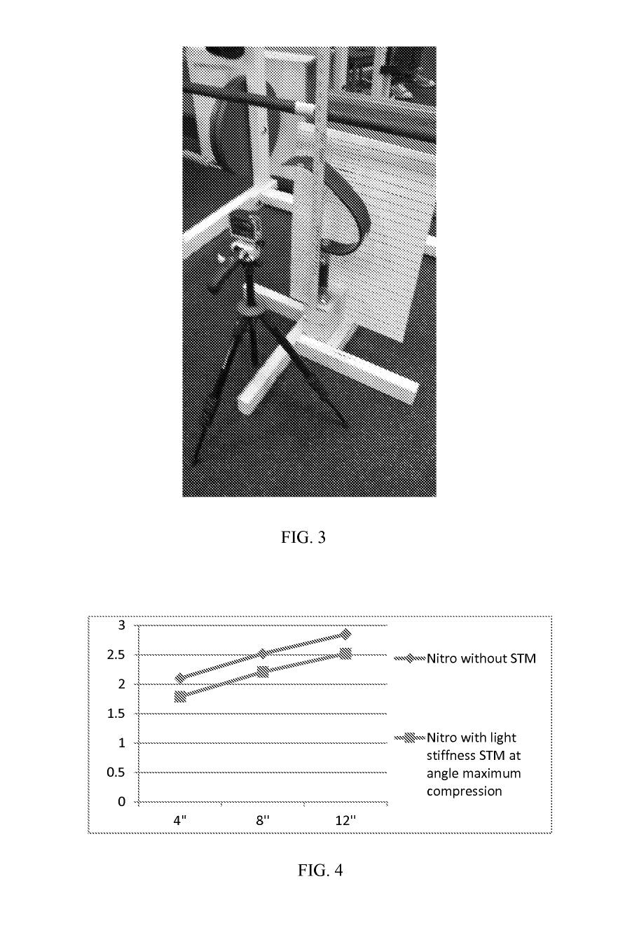

The amount of deflection in the NITRO® prosthetic with and without an attachment member comprising a STM is plotted on a line graph in FIG. 4. The graph shows that the NITRO® prosthetic with an attachment member comprising a light stiffness STM had smaller amounts of compression at all drop heights compared to the NITRO® prosthetic without an attachment member comprising a STM. There is a very slight decrease in slope from 8 to 12 inches with the NITRO® prosthetic with an attachment member comprising a light stiffness STM.

The amount of energy return/deflection of the NITRO® prosthetic with and without an attachment member comprising a STM is plotted on a scatter plot in FIG. 5. The scatter plot shows that the NITRO® prosthetic with an attachment member comprising a light stiffness STM has data points either on or above the trend line, whereas the NITRO® prosthetic without an attachment member comprising a STM has data points on or below the trend line.

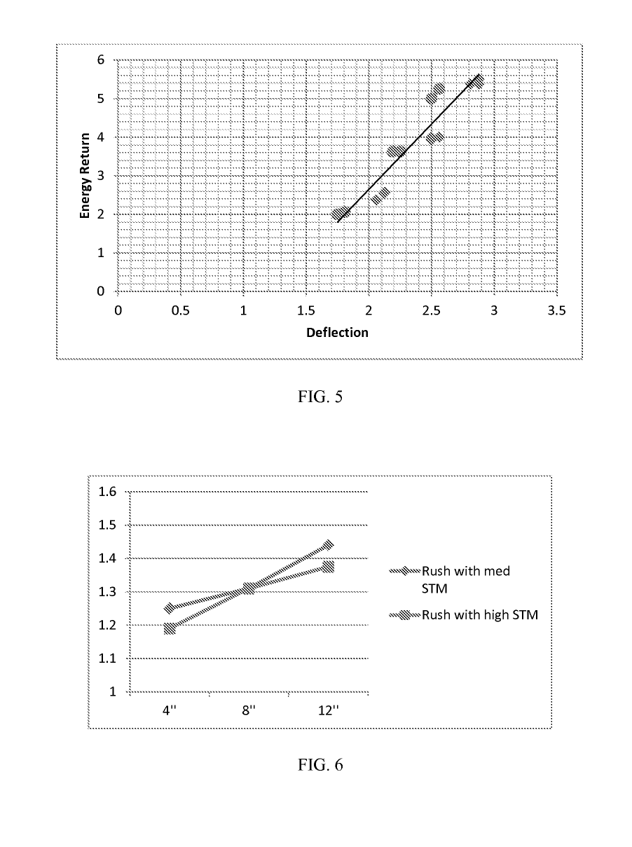

The amount of deflection in the RUSH® 87 prosthetic with an attachment member comprising medium and high stiffness STMs is plotted in a line graph in FIG. 6. The line graph shows that the RUSH® 87 prosthetic with an attachment member comprising a high stiffness STM had decreased deflection at 4 and 12 inches, as well as a leveling of the slope in the line from 8 to 12 inches.

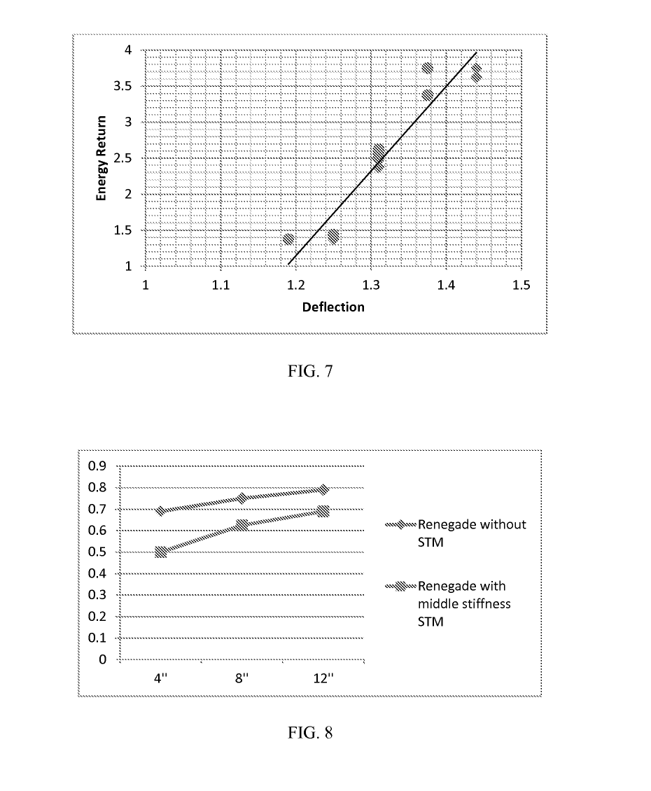

The amount of energy return/deflection in the RUSH® 87 prosthetic with an attachment member comprising medium and high stiffness STMs is plotted in a scatter plot in FIG. 7. The scatter plot shows that the RUSH® 87 prosthetic with an attachment member comprising a high stiffness STM has data points all on or above the trend line, with the exception of one data point, whereas the RUSH® 87 prosthetic with an attachment member comprising a medium stiffness STM has data points all on or below the trend line.

The amount of deflection in the RENEGADE® prosthetic with and without an attachment member comprising a medium stiffness STM is plotted on a line graph in FIG. 8. The line graph shows that the RENEGADE® prosthetic with an attachment member comprising a medium stiffness STM has decreased deflection at all drop heights with a leveling of the slope of the line from 8 to 12 inches.

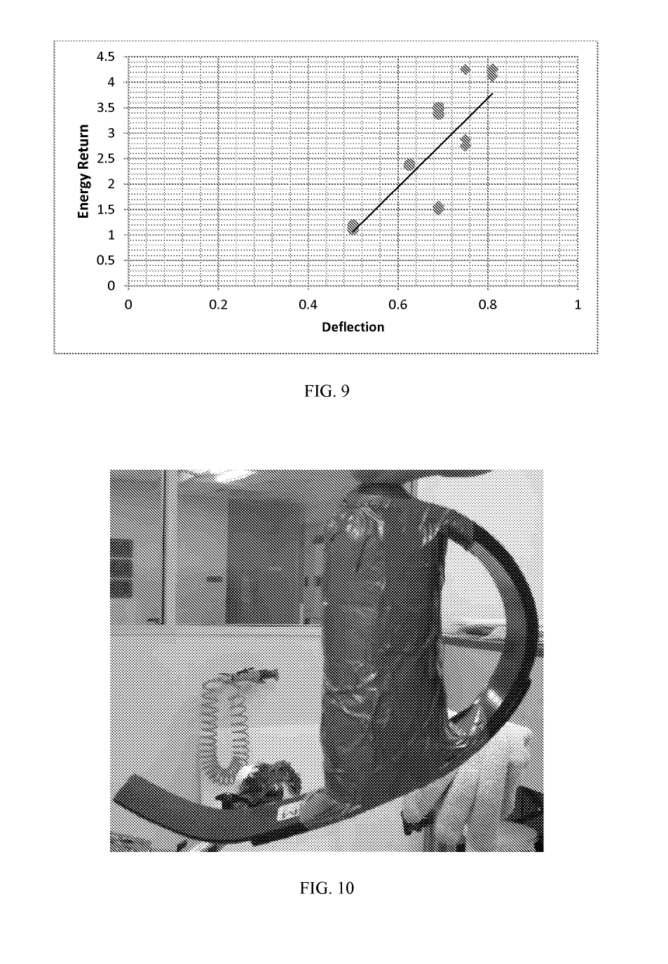

The amount of energy return/deflection in the RENEGADE® prosthetic with and without an attachment member comprising a medium stiffness STM is plotted in a scatter plot in FIG. 9. The scatter plot shows that the RENEGADE® prosthetic with an attachment member comprising a medium stiffness STM has data points all on or above the trend line, whereas the RENEGADE® prosthetic without an attachment member comprising a STM has data points all on or below the trend line with the exception of the 12 inch drop height data points that are above the trend line.

### 4. Compressibility Test

Different STMs, provided without description by ZB Products, L.P. of Katy, Tex., were tested for their compressibility at varying impact speeds. The speeds represent heel strike speeds from slow walking to jogging. A pendulum with an impact arm at the end was raised to a predetermined height and then released. The pendulum would swing and impact the STMs. The starting pendulum height was adjusted to recreate impact speeds of 0.8 m/s, 1.57 m/s, 1.96 m/s, 2.35 m/s, and 2.89 m/s. The pendulum was dropped without any excess weight or with a 30 lb lead block attached on the end of the pendulum.

The STM was encapsulated on all sides, except the impacting surface. The STM was a cylinder of 1″ in height and 1.5″ in diameter. The impact speed was measured using a high speed camera and computer software as described above. The impact deformation was calculated using physical measurements, high speed cameras, and computer software. The pendulum was dropped 3 times from each height and the three results were averaged. There was a resting period between each drop, and the material was reset to its original state. The results of compressibility test is depicted in FIG. 19.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A prosthetic foot comprising: a heel member, a toe member, and an attachment member comprising a shear thickening material (STM).

2. The prosthetic foot of claim 1, wherein the attachment member has a first end and a second end.

3. The prosthetic foot of claim 2, wherein the first end is connected to the heel member, and the second end is connected to the toe member.

4. The prosthetic foot of claim 1, wherein the attachment member further comprises an elastic polymer.

5. The prosthetic foot of claim 4, wherein the elastic polymer is impregnated with the STM.

6. The prosthetic foot of claim 1, wherein the attachment member further comprises an elastic reservoir.

7. The prosthetic foot of claim 6, wherein the elastic reservoir contains the STM.

8. The prosthetic foot of claim 1, wherein the attachment member further comprises a rigid reservoir.

9. The prosthetic foot of claim 8, wherein the rigid reservoir contains the STM.

10. The prosthetic foot of claim 1, wherein the attachment member is the lamina of the prosthetic foot.

11. The prosthetic foot of claim 1, wherein the attachment member further comprises a rigid sealed container containing the STM and a piston.

12. The prosthetic foot of claim 11, wherein the piston is connected to the heel member.

13. The prosthetic foot of claim 1, wherein the STM is a mixture of silica and polyethylene glycol.

14. The prosthetic foot of claim 1, wherein the STM is selected from silicone polymers exhibiting shear thickening properties.

15. The prosthetic foot of claim 14, wherein the silicone polymer is selected from borated silicone polymers.

16. The prosthetic foot of claim 15, wherein the borated silicone polymer is polyborodimethylsiloxane (PBDMS).

17. The prosthetic foot of claim 1, wherein the attachment member comprises 50-100% STM by weight.

18. The prosthetic foot of claim 1, wherein the attachment member comprises 65-95% STM by weight.

19. The prosthetic foot of claim 1, wherein the attachment member comprises 70% STM by weight.

20. The prosthetic foot of claim 1, further comprising an outer shell.
